(19) United States
(12) Patent Application Publication
(10) Pub. No.: US 2025/0242090 A1
(43) Pub. Date: Jul. 31, 2025

(54) METHOD FOR MANUFACTURING FUNCTIONAL ELONGATE INSTRUMENT AND FUNCTIONAL ELONGATE INSTRUMENT

(71) Applicant: TOHOKU UNIVERSITY, Sendai-shi, Miyagi (JP)

(72) Inventors: Yuanyuan GUO, Sendai-shi (JP); Yuichi SATO, Sendai-shi (JP)

(21) Appl. No.: 18/855,053

(22) PCT Filed: Apr. 13, 2023
(86) PCT No.: PCT/JP2023/015047

§ 371 Date: Oct. 8, 2024

(30) Foreign Application Priority Data
Apr. 13, 2022 (WO) PCT/JP2022/017664

(51) Int. Cl. A61L 29/12; A61B 17/00; A61B 18/00; A61B 18/24; A61L 29/02; A61L 29/08; A61M 25/00; A61M 25/01

(52) U.S. Cl. CPC A61L 29/123; A61B 18/24; A61L 29/02; A61L 29/085; A61B 2017/00526; A61B 2017/00867; A61B 2017/00955; A61B 2018/00083; A61L 2400/16; A61M 25/0009; A61M 25/0158

(57) ABSTRACT

A method for manufacturing a functional elongate instrument includes: a base material preparation step of obtaining a wire-inserted base material by inserting a first wire as an actuator member through a first through-hole of a plurality of through-holes formed in a base material and inserting a second wire opposite to the first wire through a second through-hole of the plurality of through-holes; a composite fiber formation step of obtaining a composite fiber by heating and drawing the wire-inserted base material while supplying the first wire and the second wire; a connection step of forming, at a tip portion of the composite fiber, a connection portion configured to electrically connect the first wire and the second wire; and an insulation step of forming an insulating layer on a surface of the connection portion.

### CROSS-REFERENCE TO RELATED APPLICATIONS

This application is the U.S. National Stage entry of International Application No. PCT/JP2023/015047, filed on Apr. 13, 2023, which claims priority to International Application No. PCT/JP2022/017664, filed on Apr. 13, 2022, both of which are hereby incorporated herein by reference in their entireties for all purposes.

### TECHNICAL FIELD

The present invention relates to a method for manufacturing a functional elongate instrument and a functional elongate instrument.

### BACKGROUND ART

There is a medical technology of moving through a branched blood vessel in a body of a patient while selecting positions in three dimensions and sending a tip of a catheter to a target point of treatment. In a typical catheter control method, a health professional controls the position of the catheter tip by moving back and forth and rotating the catheter outside the body of the patient. Active catheters facilitating the tip position control by adding an actuator function to the catheter have been reported. Actuator components used include a shape-memory alloy (SMA).

Patent document 1 discloses a steerable catheter or endoscope structure for observing or treating a screened subject accessible via a narrow and/or sinuous passage. The structure includes an elastically deformable longitudinal body including at least one actuator made of shape-memory material and incorporated longitudinally in the longitudinal body together with a Joule-effect heater means enabling the actuator to contract longitudinally to cause the longitudinal body to bend. The actuator extends over at least part of the longitudinal body whose rigidity varies.

Patent Document 2 discloses a medical elongate body including a portion formed of polymer material containing a laser color former on at least part of a surface of the medical elongate body and including a color forming portion formed by color forming of the laser color former by laser beam irradiation on the portion.

### CITATION LIST

### Patent Document

Patent Document 1: JP 2009-104121 A
Patent Document 2: JP 2002-136600 A

### SUMMARY OF INVENTION

### Technical Problem

Unfortunately, the technique using the actuator of Patent Document 1 makes the wire diameter 2 mm to 6 mm, which is larger than the diameters of known catheters and may have difficulty in inserting the catheter into a capillary vessel or the like.

In the actuator technique of Patent Document 2, the core wire of the shape-memory alloy is covered by extrusion molding, failing to incorporate a functional member such as a conductive material into a functional elongate instrument while maintaining the diameter. In the related art, a function of a sensor or the like cannot be incorporated in addition to the actuator at a diameter of 1 mm or less, and it may be difficult to simultaneously cover a plurality of core materials having different thermoplasticity while maintaining the positional relationship therebetween.

The present invention has been made in view of the above-described circumstances, and an object thereof is to provide a method for manufacturing a functional elongate instrument and a functional elongate instrument that is multifunctional and can have a diameter of 1 mm or less.

### Solution to Problem

The gist of the present invention is as follows.

(1) A method for manufacturing a functional elongate instrument according to a first aspect of the present invention includes: a base material preparation step of obtaining a wire-inserted base material by inserting a first wire as an actuator member through a first through-hole of a plurality of through-holes formed in a base material and inserting a second wire opposite to the first wire through a second through-hole of the plurality of through-holes; a composite fiber formation step of obtaining a composite fiber by heating and drawing the wire-inserted base material while supplying the first wire and the second wire; a connection step of forming, at a tip portion of the composite fiber, a connection portion configured to electrically connect the first wire and the second wire; and an insulation step of forming an insulating layer on a surface of the connection portion.

(2) A second aspect of the present invention is the method for manufacturing a functional elongate instrument according to the first aspect. In the composite fiber formation step, a heating temperature during the heating and drawing may be in a temperature range from 180° C. to 400° C.

(3) A third aspect of the present invention is the method for manufacturing a functional elongate instrument according to the first or second aspect. The base material may be made of thermoplastic resin.

(4) A fourth aspect of the present invention is the method for manufacturing a functional elongate instrument according to the third aspect. The thermoplastic resin may be polycarbonate, polystyrene, polyetherimide, or polysulfone.

(5) A fifth aspect of the present invention is the method for manufacturing a functional elongate instrument according to any one of the first to fourth aspects. The base material may include a plastic conductive member inside the base material. In the base material preparation step, a third wire opposite to the plastic conductive member may be inserted through a third through-hole of the plurality of through-holes.

(6) A sixth aspect of the present invention is the method for manufacturing a functional elongate instrument according to the fifth aspect and may further include, in the base material preparation step, an electrode formation step of forming an electrode containing AgCl at the tip portion of the composite fiber and at a tip of the third wire. The third wire may be made of Ag.

(7) A seventh aspect of the present invention is the method for manufacturing a functional elongate instrument according to any one of the first to fourth aspects. In the base material preparation step, a third wire as a conductive wire rod may be inserted through a third through-hole of the plurality of through-holes, and a fourth wire opposite to the third wire may be inserted through a fourth through-hole of the plurality of through-holes.

(8) An eighth aspect of the present invention is the method for manufacturing a functional elongate instrument according to any one of the first to seventh aspects. A diameter of the composite fiber may range from 0.1 mm to 1 mm.

(9) A ninth aspect of the present invention is the method for manufacturing a functional elongate instrument according to any one of the first to eighth aspects. The actuator member may be a shape-memory alloy.

(10) A tenth aspect of the present invention is the method for manufacturing a functional elongate instrument according to the ninth aspect. The shape-memory alloy may be a bendable shape-memory alloy.

(11) An eleventh aspect of the present invention is the method for manufacturing a functional elongate instrument according to the tenth aspect. A bending angle of the bendable shape-memory alloy may range from 0° to 180°.

(12) A twelfth aspect of the present invention is the method for manufacturing a functional elongate instrument according to the tenth aspect. A bending angle of the bendable shape-memory alloy may range from 0° to 20°.

(13) A functional elongate instrument according to a thirteenth aspect of the present invention includes a composite fiber and a tip portion at one end portion of the composite fiber. The composite fiber includes a first wire as an actuator member, a second wire opposite to the first wire, and an insulating portion configured to cover the first wire and the second wire. The tip portion includes a connection portion configured to electrically connect the first wire and the second wire. A diameter of the composite fiber ranges from 0.1 mm to 1 mm or less.

(14) A fourteenth aspect of the present invention is the functional elongate instrument according to the thirteenth aspect. The actuator member may be a shape-memory alloy.

(15) A fifteenth aspect of the present invention is the functional elongate instrument according to the fourteenth aspect. The actuator member may be a bendable shape-memory alloy.

(16) A sixteenth aspect of the present invention is the functional elongate instrument according to the fifteenth aspect. A bending angle of the bendable shape-memory alloy may range from 0° to 180°.

(17) A seventeenth aspect of the present invention is the functional elongate instrument according to the fifteenth aspect. A bending angle of the bendable shape-memory alloy may range from 0° to 20°.

(18) An eighteenth aspect of the invention is the functional elongate instrument according to the fifteenth aspect and may further include a plastic conductive wire rod covered with the insulating portion and a third wire that is covered with the insulating portion and is opposite to the plastic conductive wire rod. The third wire may be made of Ag. The third wire may include an electrode containing AgCl at a tip thereof.

(19) A nineteenth aspect of the invention is the functional elongate instrument according to the fifteenth aspect and may further include a third wire covered with the insulating portion and a fourth wire that is covered with the insulating portion and is opposite to the third wire.

(20) A twentieth aspect of the present invention is the functional elongate instrument according to any one of the thirteenth to nineteenth aspects. The insulating portion may be made of thermoplastic resin.

(21) A twenty-first aspect of the present invention is the functional elongate instrument according to the twentieth aspect. The thermoplastic resin may be polycarbonate, polystyrene, polyetherimide, or polysulfone.

(22) A twenty-second aspect of the present invention is the functional elongate instrument according to any one of the thirteenth to twenty-first aspects and may further include a hollow portion extending in a longitudinal direction of the composite fiber.

### Advantageous Effects of Invention

The above-described aspects of the present invention can provide a method for manufacturing a functional elongate instrument and a functional elongate instrument, where the functional elongate instrument is multifunctional and can have a diameter of 1 mm or less.

### BRIEF DESCRIPTION OF DRAWINGS

FIG. 1 is a schematic view of a functional elongate instrument according to a first embodiment.

FIG. 2 is a cross-sectional view of the functional elongate instrument taken along a line A-A in FIG. 1.

FIG. 3 is an enlarged perspective view of a tip portion of the functional elongate instrument in FIG. 1.

FIG. 4 is a cross-sectional view of a functional elongate instrument according to a modification of the first embodiment.

FIG. 5 is a flowchart of a method for manufacturing the functional elongate instrument according to the first embodiment.

FIG. 6 is a schematic view of a wire-inserted base material.

FIG. 7 is a diagram for explaining a process of heating and drawing.

FIG. 8 is a schematic view of a wire-inserted base material in a modification.

FIG. 9 is a schematic view of a functional elongate instrument according to a second embodiment.

FIG. 10 is a cross-sectional view of the functional elongate instrument taken along a line B-B in FIG. 9.

FIG. 11 is an enlarged perspective view of a tip portion of the functional elongate instrument in FIG. 9.

FIG. 12 is a flowchart of a method for manufacturing the functional elongate instrument according to the second embodiment.

FIG. 13 is a diagram for explaining a process of heating and drawing in the second embodiment.

FIG. 14 shows cross-sectional photographs of functional elongate instruments of Examples.

FIG. 15 is a diagram for explaining a method of measuring displacement.

FIG. 16 illustrates graphs each depicting a relationship between displacement of a functional elongate instrument and time in each Example.

FIG. 17 is a graph depicting a relationship between a current and time when an adrenaline concentration is varied.

FIG. 18 is a graph depicting a relationship between a bending angle and driving energy of a functional elongate instrument of a First Example.

FIG. 19 is a graph depicting a relationship between a bending angle and driving energy of a functional elongate instrument of a Second Example.

FIG. 20 is a graph depicting a relationship between a bending angle and driving energy of a functional elongate instrument of a Third Example.

FIG. 21 is a graph depicting a relationship between a bending angle and driving energy of a functional elongate instrument of a Fourth Example.

### DESCRIPTION OF EMBODIMENT

### First Embodiment

Hereinafter, an embodiment will be described with reference to the drawings. In the drawings used in the following description, to facilitate the understanding of the features, portions corresponding to the features are sometimes illustrated being enlarged for the sake of convenience and the dimensional ratios and the like of constituent elements may differ from the actual ones. Materials, dimensions, and the like exemplified in the following description are examples; the present invention is not limited thereto, and can be appropriately modified and implemented within a range where the advantageous effects of the present invention are exhibited.

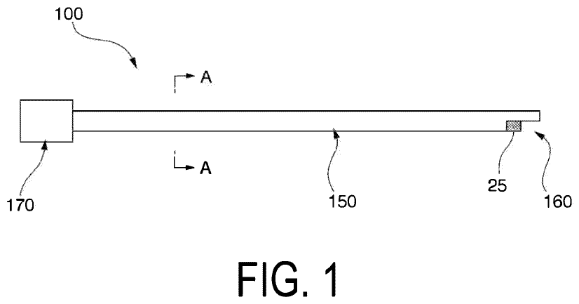

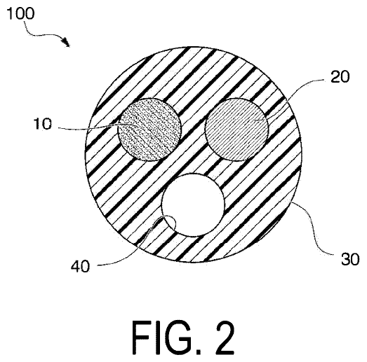

FIG. 1 illustrates a schematic view of a functional elongate instrument 100 according to the present embodiment. FIG. 2 is a cross-sectional view of the functional elongate instrument taken along a line A-A in FIG. 1. The functional elongate instrument 100 according to the first embodiment includes a composite fiber 150, a tip portion 160 located at one end portion of the composite fiber 150, and a connector portion 170 located at the other end portion. Each constituent portion will be described below.

### Composite Fiber

The composite fiber 150 includes a first wire 10 as an actuator member, a second wire 20 opposite to the first wire 10, an insulating portion 30 covering the first wire 10 and the second wire 20, and a hollow portion 40.

The length of the composite fiber 150 is not limited to a particular length. The length of the composite fiber 150 can be appropriately adjusted in accordance with an application such as a catheter.

The cross-sectional shape of the composite fiber 150 is not limited to a particular shape. The cross-sectional shape of the composite fiber 150 is, for example, a polygonal shape, a circular shape, or an elliptical shape.

The diameter of the composite fiber 150 (the longest length between vertexes when the cross-sectional shape of the composite fiber 150 is a polygonal shape or the length of the major axis when the cross-sectional shape of the composite fiber 150 is an elliptical shape) ranges from 0.1 mm to 1 mm. Setting the diameter in a range from 0.1 mm to 1 mm makes it easy to enter a blood vessel.

### First Wire

The first wire 10 is formed of an actuator member. The actuator member is, for example, a shape-memory alloy. Examples of the shape-memory alloy used for the first wire 10 include an expansion/contraction type shape-memory alloy that expands and contracts when heated and a bendable type shape-memory alloy that bends when heated. In this case, the expansion/contraction type shape-memory alloy is a shape-memory alloy in which the shape memory is a straight line (extension). The bendable type shape-memory alloy is a shape-memory alloy in which the shape memory is a curved line (bend).

As the first wire 10, in the case of the expansion/contraction type, there is a possibility that the first wire 10 is peeled from the insulating portion 30 due to expansion/contraction of the first wire 10 by heating, and therefore the bendable type shape-memory alloy is preferable. The shape-memory alloy can be controlled by, for example, Joule heat generated by a voltage being applied from the connector portion 170. A maximum surface temperature when covered with the insulating portion 30 is, for example, 40° C. to 45° C.

In the case where the first wire 10 is a bendable shape-memory alloy, it is preferable that the bendable shape-memory alloy be a shape-memory alloy in which the bending angle is memorized in advance to range from 0° to 180°. More preferably, the bendable shape-memory alloy is a shape-memory alloy in which the bending angle is memorized in advance to range from 0° to 20°. When the bending angle is large, bending performance is enhanced, but the member is significantly deformed during thermal drawing treatment, thereby making it difficult to fabricate the functional elongate instrument 100.

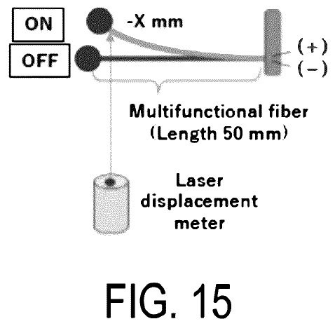

Assuming that a fiber length is L, a bending deformation quantity is x, and bending deformation is along a circumference with a radius of curvature R, a bending angle θ is represented by the following equation (1). As illustrated, for example, in FIG. 15, the bending deformation quantity can be obtained by applying a voltage to the functional elongate instrument 100 and measuring the displacement thereof with a laser displacement meter (for example, LK-G3000 manufactured by KEYENCE CORPORATION).

$$tan θ = \frac{2 Lx}{{L}^{2} - {x}^{2}} \qquad [ Math 1 ]$$

### Method of Memorizing Bending Angle of Bendable Shape-Memory Alloy

To memorize the designed curvature shape, a shape-memory alloy wire is fixed by an aluminum pipe having a predetermined curvature and is subjected to heat treatment at a temperature in a range from 400° C. to 500° C. A predetermined bending angle can be memorized by deforming the shape-memory alloy wire after the heat treatment into a linear shape.

The diameter of the first wire 10 is not limited as long as the diameter of the composite fiber 150 can range from 0.1 mm to 1 mm. The diameter of the first wire 10 is, for example, 0.05 mm to 0.3 mm. For example, when a diameter φ of the composite fiber 150 is 0.2 mm, seven members (for example, seven first wires 10) each having a diameter of 0.05 mm can be introduced.

### Second Wire

The second wire 20 is opposite to the first wire 10. The material of the second wire 20 is not limited to a particular material, and examples thereof include stainless steel, copper, and the same shape-memory alloy as that of the first wire.

The diameter of the second wire 20 is not limited as long as the diameter of the composite fiber 150 can range from 0.1 mm to 1 mm or less. The diameter of the second wire 20 is, for example, 0.05 mm to 0.3 mm.

### Insulating Portion

The insulating portion 30 covers the conductive wire rods such as the first wire 10 and the second wire 20 inside the composite fiber 150, and insulates the conductive wire rods from each other. In the first embodiment, the first wire 10 and the second wire 20 are insulated from each other. The material of the insulating portion 30 is, for example, thermoplastic resin that can be heated and drawn. The thermoplastic resin used for the insulating portion 30 is, for example, polycarbonate, polystyrene, polyetherimide, or polysulfone.

### Hollow Portion 40

The hollow portion 40 extends in the longitudinal direction of the composite fiber 150. Due to the presence of the hollow portion 40, for example, it is possible to flow a liquid medicine or the like into the hollow portion 40 from an injection port (not illustrated) provided at the connector portion 170 side, and to apply the liquid medicine to the target affected part through the tip portion 160. The diameter and shape of the hollow portion 40 can be appropriately set in accordance with the purpose.

### Tip Portion

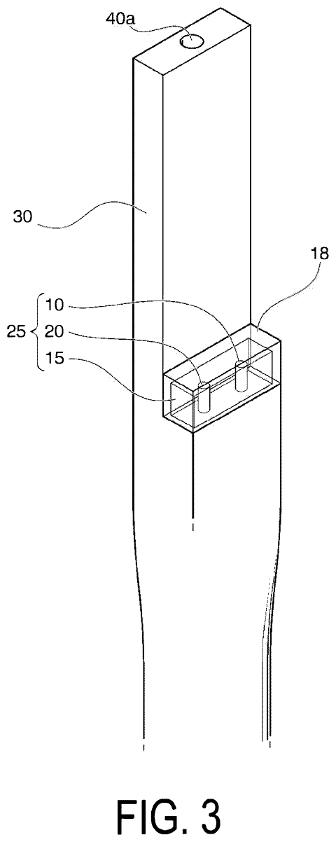

FIG. 3 is an enlarged perspective view of the tip portion 160 of the functional elongate instrument 100 in FIG. 1. The tip portion 160 includes an actuation portion 25 and an opening 40a. The actuation portion 25 includes the first wire 10, the second wire 20, a connection portion 15 for electrically connecting the first wire 10 and the second wire 20, and an insulating layer 18 covering a surface of the connection portion 15. By applying a voltage between the first wire 10 and second wire 20 of the actuation portion 25, the actuation portion 25 moves due to deformation of the first wire, and the tip position of the composite fiber 150 can be controlled.

### Connection Portion

The connection portion 15 electrically connects the first wire 10 and the second wire 20. The material of the connection portion 15 is not limited as long as the first wire 10 and the second wire 20 can be electrically connected to each other. The material thereof is Ag, for example. The first wire 10 and the second wire 20 are connected by the connection portion 15, and a voltage can be applied between the first wire 10 and the second wire 20. As a result, the actuation portion 25 can be operated.

### Insulating Layer

The insulating layer 18 is a layer made of an insulator and covers the connection portion 15. The presence of the insulating layer 18 makes it possible to prevent an electric shock when the actuator member operates.

### Opening

The opening 40a communicates with the hollow portion 40. A liquid medicine or the like entering from an injection port (not illustrated) of the connector portion 170 passes through the hollow portion 40 and is emitted from the opening 40a. With this, for example, the liquid medicine can be applied to the target affected part.

### Connector Portion

The connector portion 170 is provided at the other end portion of the composite fiber 150. The connector portion 170 includes a connector (not illustrated) for connecting the first wire 10 and second wire 20 to an external device. In addition, the injection port for injecting a liquid medicine or the like into the hollow portion 40 is provided.

The functional elongate instrument 100 according to the present embodiment has been described above. The functional elongate instrument 100 according to the present embodiment has a diameter of 1 mm or less, and has a liquid medicine injection function in addition to a tip position control function by the actuator.

In the present embodiment, there is one actuation portion 25 including the first wire 10, the second wire 20, and the connection portion 15. However, there may be two or more actuation portions 25 each including the first wire 10, the second wire 20, and the connection portion 15.

In the present embodiment, the functional elongate instrument 100 includes the hollow portion 40 but may include an optical fiber instead. By the irradiation of a laser beam from the optical fiber, the instrument can be used for ablation treatment.

Although the hollow portion 40 is provided in the present embodiment, the functional elongate instrument 100 may be provided with a conductive wire rod instead. As the conductive wire rod, a non-plastic conductive material may be used. Examples of the non-plastic conductive material include a metal wire rod, an alloy wire rod, and a carbon wire rod. In place of the hollow portion 40, a pair of metal wires may be added. When a pair of metal wires made of different metals is provided, a function of a thermocouple type temperature sensor can be added. Providing a heating element such as a pair of nichrome wires allows a function of a heater to be added, allowing the instrument to be used for ablation treatment. Furthermore, by using a pair of platinum (Pt) wires or a pair of tungsten (W) wires, a function of applying electrical stimulation to living tissue can be added.

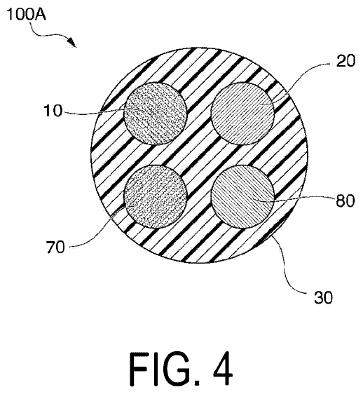

Although the first wire 10 and the second wire 20 are used in the present embodiment, the present invention is not limited thereto. FIG. 4 is a cross-sectional view of a functional elongate instrument 100A according to a modification of the first embodiment. The functional elongate instrument 100A includes the first wire 10, the second wire 20, a third wire 70 covered with the insulating portion 30, and a fourth wire 80 that is covered with the insulating portion 30 and is opposite to the third wire. The third wire 70 and the fourth wire 80 are, for example, conductive wire rods.

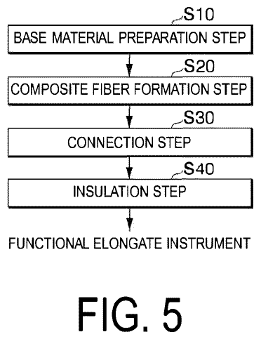

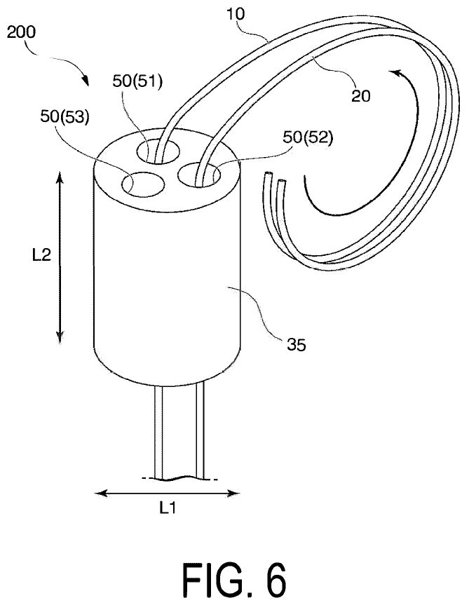

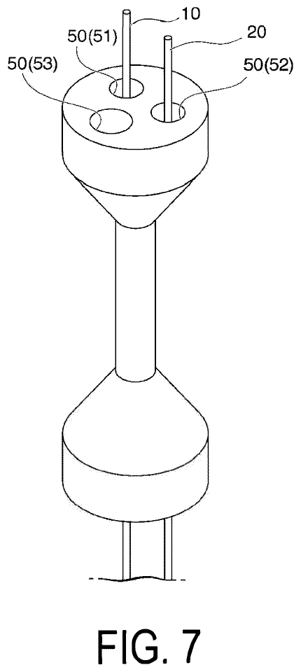

Next, a method for manufacturing the functional elongate instrument according to the present embodiment will be described. FIG. 5 is a flowchart of the method for manufacturing the functional elongate instrument according to the present embodiment. FIG. 6 is a schematic view of a wire-inserted base material. FIG. 7 is a schematic view of a composite fiber formation step by heating and drawing. The method includes a base material preparation step S10 for obtaining a wire-inserted base material 200 by inserting the first wire 10 as an actuator member through a first through-hole 51 of a plurality of through-holes 50 formed in a base material 35 of the present embodiment and inserting the second wire 20 opposite to the first wire 10 through a second through-hole 52 of the plurality of through-holes 50; a composite fiber formation step S20 for obtaining the composite fiber 150 by heating and drawing the wire-inserted base material 200 while supplying the first wire 10 and the second wire 20; a connection step S30 for forming the connection portion 15 configured to electrically connect the first wire and the second wire at the tip portion 160 of the composite fiber 150; and an insulation step S40 for forming the insulating layer 18 on a surface of the connection portion 15.

### Base Material Preparation Step

In the base material preparation step S10, the first wire 10 as the actuator member is inserted through the first through-hole 51 of the plurality of through-holes 50 formed in the base material 35, and the second wire 20 opposite to the first wire 10 is inserted through the second through-hole 52 of the plurality of through-holes 50, thereby obtaining the wire-inserted base material 200.

### Base Material

The plurality of through-holes 50 are formed in the base material 35. In the present embodiment, the base material 35 includes the first through-hole 51, through which the first wire 10 is inserted, the second through-hole 52, through which the second wire 20 is inserted, and a hollow portion through-hole 53, which serves as the hollow portion 40. The number of through-holes 50 can be appropriately set in accordance with the number and functions of wires to be incorporated into the composite fiber 150.

The cross-sectional shape of the through-hole 50 taken along a plane perpendicular to the longitudinal direction of the base material 35 is not limited to a particular shape. The cross-sectional shape of the through-hole 50 is, for example, a circle, an ellipse, or a polygon. The diameter of the through-hole 50 (the longest length between vertexes when the cross-sectional shape of the through-hole 50 is a polygonal shape or the length of the major axis when the cross-sectional shape of the through-hole 50 is an elliptical shape) can be approximately set in accordance with the diameter of the wire to insert and the diameter of the hollow portion 40 to form. The diameter of the through-hole 50 is, for example, 0.5 mm to 3 mm.

The outer peripheral shape of the base material 35 taken along a plane perpendicular to the longitudinal direction of the base material 35 is not limited to a particular shape. The outer peripheral shape of the base material 35 is, for example, a circular shape, an elliptical shape, or a polygonal shape.

A diameter L1 of the base material 35 (the longest length between vertexes when the cross-sectional shape of the base material 35 is a polygonal shape or the length of the major axis when the cross-sectional shape of the base material 35 is an elliptical shape) can be approximately set in accordance with the diameter of the composite fiber 150. The diameter L1 of the base material 35 is, for example, 5 mm to 50 mm.

A length L2 of the base material 35 can be appropriately set in accordance with a target length of the composite fiber 150. Neither the volume of the base material 35 nor the volume of the composite fiber 150 changes before and after the thermal drawing. In a case where the composite fiber 150 with a diameter of 1 mm is manufactured to have a length of 1 m, the length L2 of the base material 35 may be, for example, 1 cm to 5 cm when the diameter L1 of the base material 35 is 5 mm to 50 mm.

The material of the base material 35 is, for example, thermoplastic resin that can be heated and drawn. The material of the base material 35 is desirably a material having a large number of characteristics as much as possible among the characteristics such as a low draw temperature able to facilitate thermal drawing with ease, flexibility able to facilitate bending motion with ease, strength with which breakage is unlikely to occur, and resistance to sterilization that uses high-temperature steam, ultraviolet rays, or the like. The thermoplastic resin used for the base material 35 is, for example, polycarbonate, polystyrene, polyetherimide, or polysulfone.

### Composite Fiber Formation Step

In the composite fiber formation step S20, the composite fiber 150 is obtained by heating and drawing the wire-inserted base material 200 while the first wire 10 and the second wire 20 being supplied. In the composite fiber formation step S20, the wire-inserted base material 200 is heated. It is preferable to perform heating for a certain period of time. The heating time is, for example, 10 minutes or more. By the base material 35 being subjected to heating, the base material 35 can be softened and drawn. The speed at which the drawing is performed (draw speed) can be appropriately set in accordance with the diameter of the base material 35 and the target diameter of the composite fiber 150. By heating and drawing, each of the through-holes becomes small, each of the wires and the base material 35 come into close contact with each other, and each wire is finally covered with the insulating portion 30. Further, the diameter of the hollow portion through-hole 53 is reduced by the heating and drawing, whereby the hollow portion through-hole 53 becomes the hollow portion 40.

In the composite fiber formation step S20, the heating temperature (for example, the setting temperature of an electric furnace) at the time of heating and drawing is preferably a temperature equal to or higher than the temperature at which the base material 35 can be drawn, and preferably a temperature at which the shape memory of the shape-memory alloy is not lost. In the composite fiber formation step S20, the heating temperature at the time of heating and drawing is preferably in a temperature range from 180° C. to 400° C. In this temperature range, the base material can be drawn without losing the shape memory of the shape-memory alloy. The lower limit of the heating temperature can be appropriately set in accordance with the softening point, the melting point, or the glass transition point of a resin used for the base material 35.

Neither the first wire 10 nor the second wire 20 stretches even when heated, and therefore the first wire 10 and the second wire 20 are supplied in accordance with the draw speed. The supply speeds of the first wire 10 and the second wire 20 are preferably equal to the draw speed of the base material 35.

### Connection Step S30

In the connection step S30, the connection portion 15 configured to electrically connect the first wire and the second wire is formed at the tip portion 160 of the composite fiber 150. A method of forming the connection portion 15 is not limited to a particular method. For example, the tip portion 160 of the composite fiber 150 is partially removed to expose the first wire 10 and the second wire 20. Subsequently, the first wire 10 and the second wire 20 may be electrically connected to each other by applying a conductive material such as Ag paste.

### Insulation Step S40

In the insulation step S40, the insulating layer 18 is formed on the surface of the connection portion 15. A method of forming the insulating layer 18 on the surface of the connection portion 15 is not limited to a particular method. For example, the insulating layer 18 may be formed by applying a curable resin to the surface of the insulating layer 18 and curing the applied curable resin.

The method for manufacturing the functional elongate instrument according to the first embodiment has been described above. The method for manufacturing the functional elongate instrument according to the first embodiment makes it possible to manufacture a functional elongate instrument having a diameter of 1 mm or less and having a liquid medicine injection function in addition to the tip position control function by the actuator.

In the base material preparation step S10 of the present embodiment, nothing is inserted through the through-hole 53, but an optical fiber, a conductive wire rod, or the like may be inserted therethrough. Examples of the conductive wire rod include a metal wire rod such as an Ag wire, which is a non-plastic conductive material, an alloy wire rod, and a carbon wire rod.

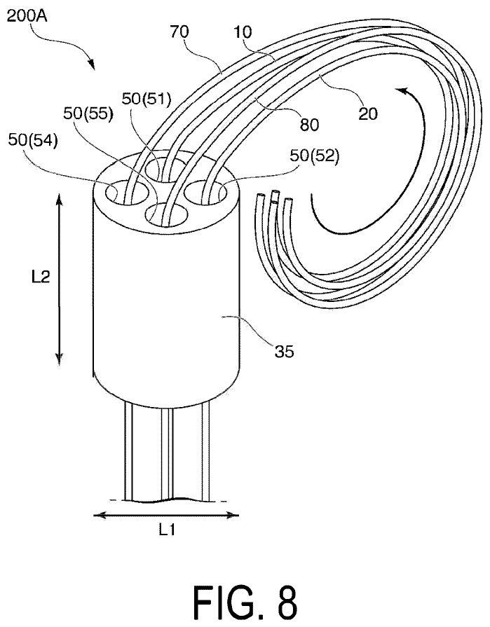

In the base material preparation step S10 of the present embodiment, the number of through-holes 50 of the base material 35 is three, but the present invention is not limited thereto. The number of through-holes 50 can be appropriately set in accordance with the number of conductive wire rods or the like to insert. FIG. 8 is a schematic view of a wire-inserted base material 200A in a modification. For example, as illustrated in FIG. 8, the third wire 70 such as an Ag wire may be inserted through a third through-hole 54 of the through-holes 50, and the fourth wire 80 opposite to the third wire 70 may be inserted through a fourth through-hole 55 of the through-holes 50. Both the third wire 70 and the fourth wire 80 may be, for example, conductive wire rods. When the third wire and the fourth wire are used in addition to the first wire 10 and the second wire 20, in the composite fiber formation step S20, the third wire 70 and the fourth wire 80 are supplied from the outside in accordance with thermal drawing deformation of the base material 35 as in the case of the first wire 10 and the second wire 20.

### Second Embodiment

Hereinafter, an embodiment will be described with reference to the drawings. In the second embodiment, the same constituent components as those in the first embodiment are denoted by the same reference signs, a description thereof is omitted, and only different points will be described.

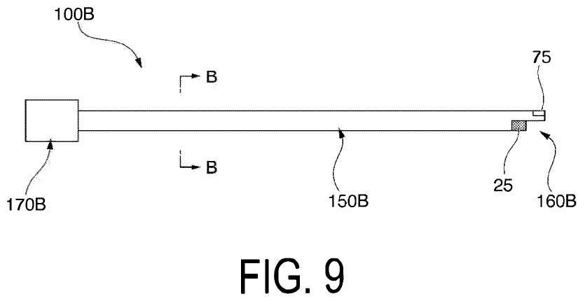

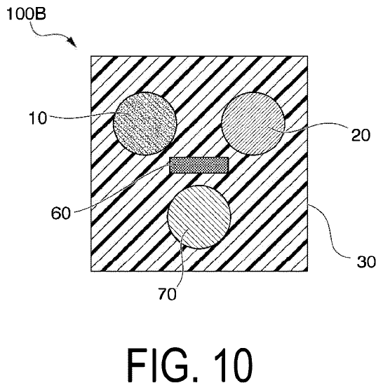

FIG. 9 illustrates a schematic view of a functional elongate instrument 100B according to the present embodiment. FIG. 10 is a cross-sectional view of the functional elongate instrument taken along a line B-B in FIG. 9. The functional elongate instrument 100B according to the second embodiment includes a composite fiber 150B, a tip portion 160B located at one end portion of the composite fiber 150B, and a connector portion 170B located at the other end portion. Each constituent portion will be described below.

### Composite Fiber

The composite fiber 150B includes a first wire 10 as an actuator member, a second wire 20 opposite to the first wire 10, an insulating portion 30 covering the first wire 10 and the second wire 20, a plastic conductive wire rod 60, and a third wire 70.

The length of the composite fiber 150B is not limited to a particular length. The length of the composite fiber 150B can be appropriately adjusted in accordance with an application such as a catheter.

The cross-sectional shape of the composite fiber 150B is not limited to a particular shape. The cross-sectional shape of the composite fiber 150B is, for example, a polygonal shape, a circular shape, or an elliptical shape.

The diameter of the composite fiber 150B (the longest length between vertexes when the cross-sectional shape of the composite fiber 150B is a polygonal shape or the length of the major axis when the cross-sectional shape of the composite fiber 150B is an elliptical shape) ranges from 0.1 mm to 1 mm. Setting the diameter in a range from 0.1 mm to 1 mm makes it easy to enter a blood vessel.

### First Wire

The first wire 10 is formed of an actuator member. The actuator member is, for example, a shape-memory alloy. As the shape-memory alloy, a bendable type shape-memory alloy (bendable shape-memory alloy) is preferable. In the case where the first wire 10 is a bendable shape-memory alloy, it is preferable that the bendable shape-memory alloy be a shape-memory alloy in which the bending angle is memorized in advance to range from 0° to 180°. It is preferable that the bendable shape-memory alloy be a shape-memory alloy in which the bending angle is memorized in advance to range from 0° to 20°. When the bending angle is large, bending performance is enhanced, but the member is significantly deformed during the thermal drawing treatment, thereby making it difficult to fabricate the functional elongate instrument 100B.

The diameter of the first wire 10 is not limited as long as the diameter of the composite fiber 150B can range from 0.1 mm to 1 mm or less. The diameter of the first wire 10 is, for example, 0.05 mm to 0.3 mm.

### Second Wire

The second wire 20 is opposite to the first wire 10. Examples of the material of the second wire 20 include stainless steel, copper, and the same shape-memory alloy as that of the first wire.

The diameter of the second wire 20 is not limited as long as the diameter of the composite fiber 150B can range from 0.1 mm to 1 mm or less. The diameter of the second wire 20 is, for example, 0.05 mm to 0.3 mm.

### Plastic Conductive Wire Rod

As the plastic conductive wire rod 60, it is preferable to use a plastic conductive material able to link with and follow the drawing deformation of the base material. Examples of the plastic conductive material include a low melting point metal material, a low melting point alloy material, a conductive paste, a metal nano-ink, and a composite material (conductive resin composite wire rod) in which an electrically conductive material is dispersed in thermoplastic resin. In particular, it is preferable to use a conductive resin composite wire rod. Examples of the electrically conductive material used for the conductive resin composite wire rod include carbon materials such as carbon nanotubes, carbon black and graphene, and metal materials such as metal particles. Carbon nanotubes are particularly preferred. Examples of the thermoplastic resin used for the conductive resin composite wire rod include polyethylene and polypropylene. The plastic conductive wire rod 60 is covered with the insulating portion 30.

The content of the electrically conductive material in the conductive resin composite wire rod is not limited as long as the conductive resin composite wire rod can have conductivity. The content of the electrically conductive material is, for example, 5 to 90 mass %. The content of the electrically conductive material is more preferably 70 mass % or less. The content of the electrically conductive material is still more preferably 50 mass % or more.

The diameter of the plastic conductive wire rod 60 is not limited as long as the diameter of the composite fiber 150B can range from 0.1 mm to 1 mm or less. The diameter of the plastic conductive wire rod 60 is, for example, 0.05 mm to 0.3 mm.

### Third Wire

The third wire 70 is opposite to the plastic conductive wire rod 60. The third wire 70 is covered with the insulating portion 30. Examples of the material of the third wire 70 include Ag, Pt, Au, and carbon.

The diameter of the third wire 70 is not limited as long as the diameter of the composite fiber 150B can range from 0.1 mm to 1 mm or less. The diameter of the third wire 70 is, for example, 0.01 mm to 0.2 mm.

### Insulating Portion

The insulating portion 30 covers the conductive wire rods inside the composite fiber 150B, and insulates the conductive wire rods from each other. In the second embodiment, the first wire 10, the second wire 20, the plastic conductive wire rod 60, and the third wire 70 are insulated from each other. The material of the insulating portion 30 is, for example, thermoplastic resin that can be heated and drawn. The thermoplastic resin used for the insulating portion 30 is, for example, polycarbonate, polystyrene, polyetherimide, or polysulfone.

### Tip Portion

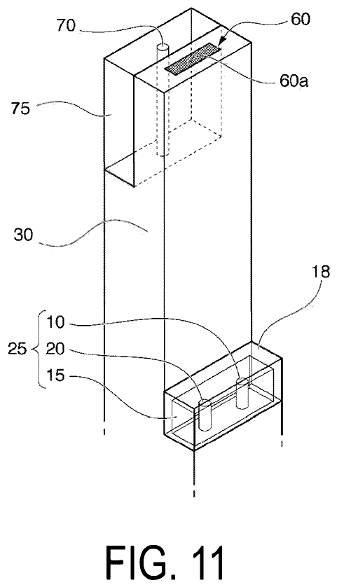

FIG. 11 is an enlarged perspective view of the tip portion 160B of the functional elongate instrument 100B in FIG. 9. The tip portion 160B includes an actuation portion 25, an exposed portion 60a, where the plastic conductive wire rod 60 is exposed, and an electrode 75. The actuation portion 25 includes the first wire 10, the second wire 20, a connection portion 15 for electrically connecting the first wire 10 and the second wire 20, and an insulating layer 18 covering a surface of the connection portion 15. By applying a voltage between the first wire 10 and second wire 20 of the actuation portion 25, the actuation portion 25 moves due to deformation of the first wire, and the tip position of the functional elongate instrument 100B can be controlled.

### Connection Portion

The connection portion 15 electrically connects the first wire 10 and the second wire 20. The material of the connection portion 15 is not limited as long as the first wire 10 and the second wire 20 can be electrically connected to each other. The material thereof is Ag, for example. The first wire 10 and the second wire 20 are connected by the connection portion 15, and a voltage can be applied between the first wire 10 and the second wire 20. As a result, the actuation portion 25 can be operated.

### Insulating Layer

The insulating layer 18 is a layer made of an insulator and covers the connection portion 15. The presence of the insulating layer 18 makes it possible to prevent an electric shock when the actuator member operates.

### Exposed Portion

The exposed portion 60a is a region where the plastic conductive wire rod 60 is exposed. The exposed portion 60a functions as, for example, a working electrode of a sensor. The area of the exposed portion 60a is not limited as long as the exposed portion 60a can function as the working electrode.

### Electrode 75

The electrode 75 is provided at a tip of the third wire. Examples of the electrode 75 include a coating film containing AgCl. The electrode 75 functions as a counter electrode to the working electrode of the sensor. The electrode 75 may function as a counter electrode and a reference electrode to the working electrode of the sensor. When the exposed portion 60a is a carbon nanotube-containing composite and the electrode 75 is a coating film containing AgCl, this configuration functions as a sensor for detecting a compound such as adrenaline.

### Connector Portion

The connector portion 170B is provided at the other end portion of the composite fiber 150B. The connector portion 170B includes a connector (not illustrated) for connecting the first wire 10, the second wire 20, the plastic conductive wire rod 60, and the third wire 70 to an external device.

The functional elongate instrument 100B according to the present embodiment has been described above. The functional elongate instrument 100B according to the present embodiment has a diameter of 1 mm or less and has a compound detection function in addition to the tip position control function by the actuator. In addition to the first wire 10, the second wire 20, the plastic conductive wire rod 60 and the third wire 70, a hollow portion and an opening that enable a liquid medicine injection function, and an optical fiber may be further provided, as in the first embodiment.

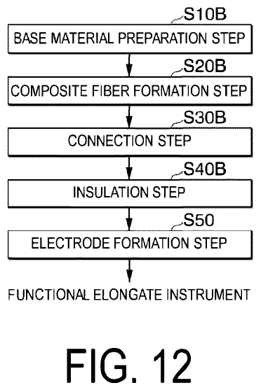

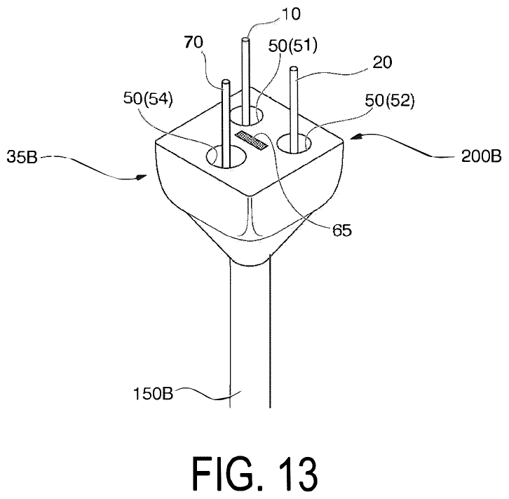

Next, a method for manufacturing the functional elongate instrument according to the second embodiment will be described. FIG. 12 is a flowchart of the method for manufacturing the functional elongate instrument according to the present embodiment. FIG. 13 is a diagram for explaining a process of heating and drawing in the second embodiment. The method includes: a base material preparation step S10B for obtaining a wire-inserted base material 200B by inserting the first wire 10 as an actuator member through a first through-hole 51 of a plurality of through-holes 50 formed in a base material 35B of the present embodiment, inserting the second wire 20 opposite to the first wire 10 through a second through-hole 52 of the plurality of through-holes 50, and inserting the third wire 70 through a third through-hole 54 of the plurality of through-holes 50; a composite fiber formation step S20B for obtaining the composite fiber 150B by heating and drawing the wire-inserted base material 200B while supplying the first wire 10, the second wire 20, and the third wire 70; a connection step S30B for forming the connection portion 15 configured to electrically connect the first wire 10 and the second wire 20 at the tip of the composite fiber 150B; an insulation step S40B for forming the insulating layer 18 on a surface of the connection portion 15; and an electrode formation step S50 for forming the electrode 75 containing AgCl at the tip portion 160B of the composite fiber 150B and at the tip of the third wire 70.

### Base Material Preparation Step

In the base material preparation step S10B, the first wire 10 as the actuator member is inserted through the first through-hole 51 of the plurality of through-holes 50 formed in the base material 35B, the second wire 20 opposite to the first wire 10 is inserted through the second through-hole 52 of the plurality of through-holes 50, and the third wire 70 is inserted through the third through-hole 54 of the plurality of through-holes 50, thereby obtaining the wire-inserted base material 200B.

### Base Material

The plurality of through-holes 50 are formed in the base material 35B. In the present embodiment, the base material 35B includes the first through-hole 51, through which the first wire 10 is inserted, the second through-hole 52, through which the second wire 20 is inserted, and the third through-hole 54, through which the third wire 70 is inserted. The number of through-holes 50 can be appropriately set in accordance with the number and functions of wires to be incorporated into the composite fiber 150B. In addition, a through-hole for a hollow portion to serve as the hollow portion 40 may be set.

FIG. 13 is a diagram for explaining a process of heating and drawing in the second embodiment. The base material 35B includes a plastic conductive member 65 inside the base material 35B. The plastic conductive member 65 is a material to become the plastic conductive wire rod 60 by being subjected to heating and drawing. A method of disposing the plastic conductive member 65 in the base material 35B is not limited to a particular method. For example, it can be disposed by a method as follows. For example, the base material 35B is cut into two pieces along a direction parallel to the longitudinal direction thereof. A groove into which the plastic conductive member 65 can be fitted is formed on a surface of the cut base material. The plastic conductive member 65 is fitted into the formed groove. By bonding the pieces of the cut base material 35B to each other by heating again or the like, it is possible to obtain the base material 35B including therein the plastic conductive member 65. The plastic conductive member 65 may use the same composition as that of the plastic conductive wire rod 60 described above.

The cross-sectional shape of the through-hole 50 taken along a plane perpendicular to the longitudinal direction of the base material 35B is not limited to a particular shape. The cross-sectional shape of the through-hole 50 is, for example, a circle, an ellipse, or a polygon. The diameter of the through-hole 50 (the longest length between vertexes when the cross-sectional shape of the through-hole 50 is a polygonal shape or the length of the major axis when the cross-sectional shape of the through-hole 50 is an elliptical shape) can be approximately set in accordance with the diameter of the wire to insert and the diameter of the hollow portion 40 to form. The diameter of the through-hole 50 is, for example, 0.5 mm to 3 mm.

The outer peripheral shape of the base material 35B taken along a plane perpendicular to the longitudinal direction of the base material 35B is not limited to a particular shape. The outer peripheral shape of the base material 35B is, for example, a circular shape, an elliptical shape, or a polygonal shape.

A diameter L1 of the base material 35B (the longest length between vertexes when the cross-sectional shape of the base material 35B is a polygonal shape or the length of the major axis when the cross-sectional shape of the base material 35B is an elliptical shape) can be approximately set in accordance with the diameter of the composite fiber 150B. The diameter L1 of the base material 35B is, for example, 5 mm to 50 mm.

A length L2 of the base material 35B can be appropriately set in accordance with the length of the composite fiber 150B. Neither the volume of the base material 35B nor the volume of the composite fiber 150B changes before and after the thermal drawing. In a case where the composite fiber 150B with a diameter of 1 mm is manufactured to have a length of 1 m, the length L2 of the base material 35B may be, for example, 1 cm to 5 cm when the diameter L1 of the base material 35B is 5 mm to 50 mm.

The material of the base material 35B is, for example, thermoplastic resin that can be heated and drawn. The material of the base material 35B is desirably a material having a large number of characteristics as much as possible among the characteristics such as a low draw temperature able to facilitate thermal drawing with ease, flexibility able to facilitate bending motion with ease, strength with which breakage is unlikely to occur, and resistance to sterilization that uses high-temperature steam, ultraviolet rays, or the like. The thermoplastic resin used for the base material 35B is, for example, polycarbonate, polystyrene, polyetherimide, or polysulfone.

### Composite Fiber Formation Step

In the composite fiber formation step S20B, as illustrated in FIG. 13, the composite fiber 150B is obtained by heating and drawing the wire-inserted base material 200B while the first wire 10, the second wire 20, and the third wire 70 are being supplied. In the composite fiber formation step S20B, the wire-inserted base material 200B is heated. By the base material 35B being subjected to heating, the base material 35B can be softened and drawn. At this time, the plastic conductive member 65 disposed inside the base material 35B is also drawn. The draw speed can be appropriately set in accordance with the diameter of the base material 35B and the target diameter of the composite fiber 150B. By heating and drawing, each of the through-holes becomes small, each of the wires and the base material 35B come into close contact with each other, and each wire is finally covered with the insulating portion 30.

In the composite fiber formation step S20B, the heating temperature at the time of heating and drawing is preferably a temperature equal to or higher than the temperature at which the base material 35B can be drawn, and preferably a temperature at which the shape memory of the shape-memory alloy is not lost. In the composite fiber formation step S20B, the heating temperature at the time of heating and drawing is preferably in a temperature range from 180° C. to 400° C. In this temperature range, the base material 35B can be drawn without losing the shape memory of the shape-memory alloy. The lower limit of the heating temperature can be appropriately set in accordance with the softening point, the melting point, or the glass transition point of a resin used for the base material 35B, or in accordance with the softening point, the melting point, or the glass transition point of thermoplastic resin of a conductive resin composite member when the conductive resin composite member is used for the plastic conductive member 65.

None of the first wire 10, the second wire 20, and the third wire 70 stretch even when they are heated, and therefore the first wire 10, the second wire 20, and the third wire 70 are supplied in accordance with the draw speed. The supply speeds of the first wire 10, the second wire 20, and the third wire 70 are preferably equal to the draw speed of the base material 35B. The supplied wires are wires that are not drawn by heating and drawing.

### Connection Step S30B

In the connection step S30B, the connection portion 15 configured to electrically connect the first wire 10 and the second wire 20 is formed at the tip portion 160B of the composite fiber 150B. A method of forming the connection portion 15 is not limited to a particular method. For example, the tip portion 160B of the composite fiber 150B is partially removed to expose the first wire 10 and the second wire 20. Subsequently, the first wire 10 and the second wire 20 may be electrically connected to each other by applying a conductive material such as Ag paste.

### Insulation Step S40B

In the insulation step S40B, the insulating layer 18 is formed on the surface of the connection portion 15. A method of forming the insulating layer 18 on the surface of the connection portion 15 is not limited to a particular method. For example, the insulating layer 18 may be formed by applying a curable resin to the surface of the insulating layer 18 and curing the applied curable resin.

### Electrode Formation Step S50

In the electrode formation step S50, the electrode 75 is formed at the tip portion 160B of the composite fiber 150B, and specifically at the tip of the third wire 70. A method of forming the electrode 75 at the tip of the third wire 70 is not limited to a particular method. For example, the tip portion 160B is partially removed to expose the tip of the third wire 70. The electrode 75 may be formed by applying a conductive paste to the exposed tip of the third wire 70. A known AgCl ink may be used for the electrode paste.

The method for manufacturing the functional elongate instrument according to the second embodiment has been described above. The method for manufacturing the functional elongate instrument according to the second embodiment can manufacture a functional elongate instrument having a diameter of 1 mm or less and having a liquid medicine injection function and a compound detection function in addition to the tip position control function by the actuator.

The technical scope of the present invention is not limited to the embodiments, and various modifications can be made without departing from the gist of the present invention. Constituent elements in the embodiments can be appropriately replaced with known constituent elements without departing from the gist of the present invention, and the constituent elements may be appropriately combined.

### EXAMPLES

Next, some examples will be described in which experiments were performed to verify the effectiveness of the method for manufacturing the functional elongate instrument and the effectiveness of the functional elongate instrument according to the present disclosure.

### Bendable Shape-Memory Alloy

As the shape-memory alloys, a shape-memory alloy wire (not memorized, 0.3 mm in diameter) manufactured by Yoshimi Inc., and WDUH2-02 (not memorized, 0.2 mm in diameter) manufactured by Actment Co., Ltd. were used. The shape-memory alloy was heated to 500° C. in a bent state, and then rapidly cooled to memorize the bent state. The shape-memory alloy was deformed into a straight line by external force, whereby a bendable shape-memory alloy was fabricated.

### First Example

A bendable shape-memory alloy was used as the first wire, a stainless steel wire (0.1 mm in diameter) was used as the second wire, an Ag wire (0.1 mm in diameter) was used as the third wire, and a 5-wt % carbon nanofiber manufactured by Sigma-Aldrich Corporation was used as a conductive resin composite wire rod for the plastic conductive wire rod 60. A polycarbonate cut plate 601004 (plate thickness: 6 mm) manufactured by Hakudo Corporation was used as a base material, 1.4-mm holes were provided as through-holes, and the first wire, the second wire, and the third wire were inserted through the respective through-holes to obtain a wire-inserted base material. The diameter of the base material was 12 mm.

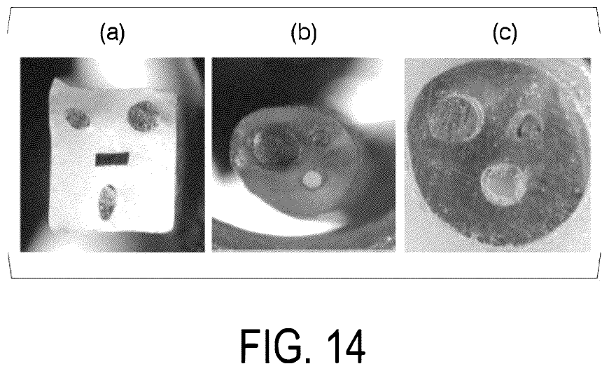

The wire-inserted base material was heated in a heating furnace at a temperature of 250° C. for 10 minutes, and was drawn using a weight of 75-g load attached to a lower portion of the base material to obtain a composite fiber. The wire-inserted base material was drawn while the respective wires were being supplied. At the tip of the obtained composite fiber, the resin was partially removed, and an Ag paste was applied to form a connection portion between the first wire and the second wire. After the connection portion was formed, a curable resin (LEM-006 manufactured by Henkel Japan Ltd.) was applied to form an insulating layer. In addition, the resin near the third wire was removed, and an Ag/AgCl ink for an AgCl ink reference electrode (silver-silver chloride ink for a reference electrode manufactured by BAS Inc.) was applied to fabricate the electrode. A cross-sectional photograph of the obtained functional elongate instrument of First Example is shown in FIG. 14(a). The diameter of the composite fiber of First Example was 1 mm or less.

### Second Example

A bendable shape-memory alloy was used as the first wire, a stainless steel wire (0.1 mm in diameter) was used as the second wire, and CK40 manufactured by Mitsubishi Chemical Corporation was used as the optical fiber. A resin-made round bar PC (polycarbonate) 2-9587 (special order product, 8 mm in wire diameter) manufactured by AS ONE Corporation was used as a base material, holes of 1.4 mm were provided as through-holes, and the first wire and the second wire were inserted through the respective through-holes to obtain a wire-inserted base material. The diameter of the base material was 8 mm.

The wire-inserted base material was heated in a heating furnace at a temperature of 250° C. for 10 minutes, and was drawn using a weight of 75-g load attached to a lower portion of the base material to obtain a composite fiber. The wire-inserted base material was drawn while the respective wires and the optical fiber being supplied. At the tip of the obtained composite fiber, the resin was partially removed, and an Ag paste was applied to form a connection portion between the first wire and the second wire. After the connection portion was formed, a curable resin (LEM-006 manufactured by Henkel Japan Ltd.) was applied to form an insulating layer. A cross-sectional photograph of the obtained functional elongate instrument of Second Example is shown in FIG. 14(b). The diameter of the composite fiber of Second Example was 1 mm or less.

### Third Example

A bendable shape-memory alloy was used as the first wire, and a stainless steel wire (0.1 mm in diameter) was used as the second wire. A resin-made round bar PC (polycarbonate) 2-9587 (special order product, 8 mm in wire diameter) manufactured by AS ONE Corporation was used as a base material. Holes of 1.4 mm were provided as through-holes (a through-hole for forming the hollow portion 40 was 2.0 mm), and the first wire and the second wire were inserted through the respective through-holes to obtain a wire-inserted base material. The diameter of the base material was 8 mm.

The wire-inserted base material was heated in a heating furnace at a temperature of 250° C. for 10 minutes, and was drawn using a weight of 75-g load attached to a lower portion of the base material to obtain a composite fiber. The wire-inserted base material was drawn while the respective wires were being supplied. At the tip of the obtained composite fiber, the resin was partially removed, and an Ag paste was applied to form a connection portion between the first wire and the second wire. After the connection portion was formed, a curable resin (LEM-006 manufactured by Henkel Japan Ltd.) was applied to form an insulating layer. A cross-sectional photograph of the obtained functional elongate instrument of Third Example is shown in FIG. 14(c). The diameter of the composite fiber of Third Example was 1 mm or less.

### Fourth Example

A bendable shape-memory alloy was used as the first wire, a stainless steel wire (0.1 mm in diameter) was used as the second wire, an Ag wire (0.1 mm in diameter) was used as the third wire, and a 5-wt % carbon nanofiber manufactured by Sigma-Aldrich Corporation was used as a conductive resin composite wire rod for the plastic conductive wire rod 60. Polyethylene (manufactured by AS ONE corporation, Product No. 2-9215-03) was used as a base material, and 1.4-mm holes were provided as through-holes. The first wire, the second wire, and the third wire were inserted through the respective through-holes to obtain a wire-inserted base material. The diameter of the base material was 12 mm.

The wire-inserted base material was heated in a heating furnace at a temperature of 250° C. for 10 minutes, and was drawn using a weight of 75-g load attached to a lower portion of the base material to obtain a composite fiber. The wire-inserted base material was drawn while the respective wires were being supplied. At the tip of the obtained composite fiber, the resin was partially removed, and an Ag paste was applied to form a connection portion between the first wire and the second wire. After the connection portion was formed, a curable resin (LEM-006 manufactured by Henkel Japan Ltd.) was applied to form an insulating layer. In addition, the resin near the third wire was removed, and an Ag/AgCl ink for an AgCl ink reference electrode (silver-silver chloride ink for a reference electrode manufactured by BAS Inc.) was applied to fabricate the electrode. A cross-sectional photograph of the obtained functional elongate instrument of First Example is shown in FIG. 14(a). The diameter of the composite fiber of First Example was 1 mm or less.

### Displacement Measurement

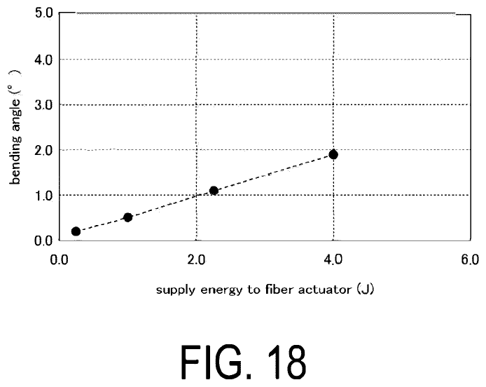

In the functional elongate instrument obtained in each Example, as illustrated in FIG. 15, a voltage was applied to the functional elongate instrument, and its displacement was measured by a laser displacement meter (LK-G3000 manufactured by Keyence Corporation). A predetermined voltage (1 to 5 V) was repeatedly applied with the electrification time of 5 seconds and the non-electrification time of 30 seconds. The bending angle was obtained based on the above-described equation (1), and the relationship between the bending angle and the driving energy was investigated. The results obtained are depicted in FIGS. 18 to 21.

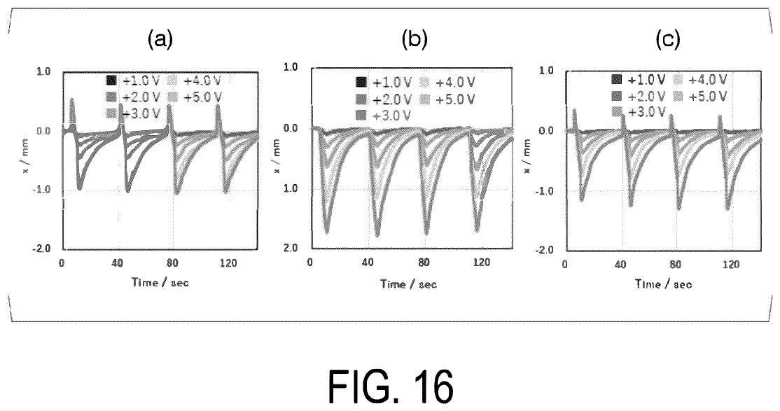

FIG. 16 illustrates graphs each depicting the relationship between time and displacement of each Example. FIG. 16(a) depicts the result of First Example, FIG. 16(b) depicts the result of Second Example, and FIG. 16(c) depicts the result of Third Example. In FIGS. 16(a), 16(b), and 16(c), the vertical axis represents the displacement x (mm) and the horizontal axis represents the time (sec). As depicted in FIGS. 16(a), 16(b), and 16(c), the functional elongate instrument of any Example was repeatedly displaced by the voltage being repeatedly applied. From the above results, it has been found that the shape memory of the shape-memory alloy was not lost in the method for manufacturing the functional elongate instrument according to the present embodiment.

### Adrenaline Amount Measurement

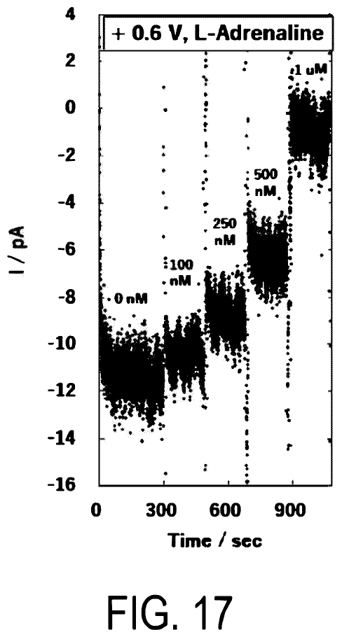

The amount of adrenaline was measured using the functional elongate instrument of First Example. The tip portion of the functional elongate instrument 100 was set in a vessel containing a phosphate buffer, and then a voltage of 0.6 V was applied between the Ag wire and the plastic conductive wire rod. Then, the adrenaline concentration was increased stepwise to 1 μM, and the amount of change in current was measured. The results obtained are depicted in FIG. 17. In FIG. 17, the vertical axis represents a current value (pA) and the horizontal axis represents time. As depicted in FIG. 17, it was found that the current value was increased by increasing the amount of adrenaline. That is, it has been confirmed that the functional elongate instrument according to the present embodiment can also measure the amount of adrenaline.

Relationship with Bending Angle

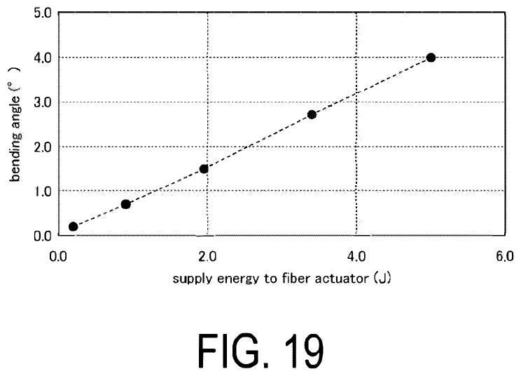

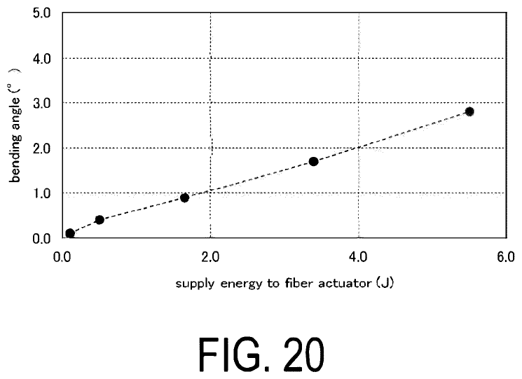

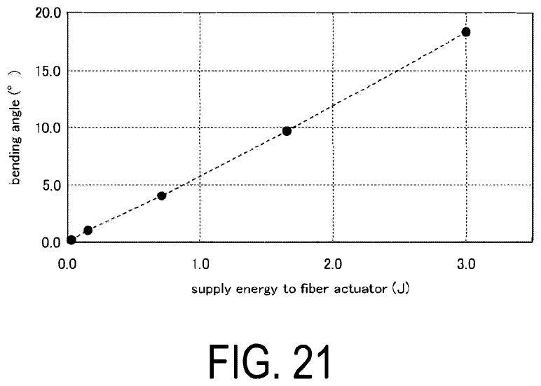

FIG. 18 is a graph depicting the relationship between the bending angle and driving energy of the functional elongate instrument of First Example. The vertical axis in FIG. 18 represents the bending angle (°), and the horizontal axis in FIG. 18 represents the driving energy (J). FIG. 19 is a graph depicting the relationship between the bending angle and driving energy of the functional elongate instrument of Second Example. The vertical axis in FIG. 19 represents the bending angle (°), and the horizontal axis in FIG. 19 represents the driving energy (J). FIG. 20 is a graph depicting the relationship between the bending angle and driving energy of the functional elongate instrument of Third Example. The vertical axis in FIG. 20 represents the bending angle (°), and the horizontal axis in FIG. 20 represents the driving energy (J). FIG. 21 is a graph depicting the relationship between the bending angle and driving energy of the functional elongate instrument of Fourth Example. The vertical axis in FIG. 21 represents the bending angle (°), and the horizontal axis in FIG. 21 represents the driving energy (J).

As depicted in FIGS. 18 to 21, it has been confirmed that the bending angle of any of the functional elongate instruments was increased by increasing the driving energy. Note that the bending angle of Fourth Example using flexible polyethylene was displaced up to 20°. It has been found that even when the maximum bending angle is 2 to 4°, fine adjustment can be performed at a level of 0.5° by power supply control, and thus the instrument can be used for sensing a sample surface or the like.

### INDUSTRIAL APPLICABILITY

The method for manufacturing a functional elongate instrument can manufacture a functional elongate instrument that is multifunctional and has a diameter of 1 mm or less, thus having a high industrial applicability.

### REFERENCE SIGNS LIST

10 First wire, 15 Connection portion, 18 Insulating layer, 20 Second wire, 30 Insulating portion, 35 Base material, 40 Hollow portion, 50 Through-hole, 51 First through-hole, 52 Second through-hole, 60 Plastic conductive wire rod, 65 Plastic conductive member, 70 Third wire, 75 Electrode, 100 Functional elongate instrument, 200 Wire-inserted base material, S10 Base material preparation step, S20 Composite fiber formation step, S30 Connection step, S40 Insulation step, S50 Electrode formation step

1. A method for manufacturing a functional elongate instrument, the method comprising:
a base material preparation step of obtaining a wire-inserted base material by inserting a first wire as an actuator member through a first through-hole of a plurality of through-holes formed in a base material and inserting a second wire opposite to the first wire through a second through-hole of the plurality of through-holes;
a composite fiber formation step of obtaining a composite fiber by heating and drawing the wire-inserted base material while supplying the first wire and the second wire;
a connection step of forming, at a tip portion of the composite fiber, a connection portion configured to electrically connect the first wire and the second wire; and
an insulation step of forming an insulating layer on a surface of the connection portion.

2. The method for manufacturing a functional elongate instrument according to claim 1, wherein in the composite fiber formation step, a heating temperature during the heating and drawing is in a temperature range from 180° C. to 400° C.

3. The method for manufacturing a functional elongate instrument according to claim 1, wherein the base material is made of thermoplastic resin.

4. The method for manufacturing a functional elongate instrument according to claim 3, wherein the thermoplastic resin is polycarbonate, polystyrene, polyetherimide, or polysulfone.

5. The method for manufacturing a functional elongate instrument according to claim 1, wherein
the base material comprises a plastic conductive member inside the base material, and
in the base material preparation step, a third wire opposite to the plastic conductive member is inserted through a third through-hole of the plurality of through-holes.

6. The method for manufacturing a functional elongate instrument according to claim 5, further comprising,
in the base material preparation step,
an electrode formation step of forming an electrode containing AgCl at the tip portion of the composite fiber and at a tip of the third wire, wherein
the third wire is made of Ag.

7. The method for manufacturing a functional elongate instrument according to claim 1, wherein
in the base material preparation step, a third wire as a conductive wire rod is inserted through a third through-hole of the plurality of through-holes, and
a fourth wire opposite to the third wire is inserted through a fourth through-hole of the plurality of through-holes.

8. The method for manufacturing a functional elongate instrument according to claim 1, wherein a diameter of the composite fiber ranges from 0.1 mm to 1 mm.

9. The method for manufacturing a functional elongate instrument according to claim 1, wherein the actuator member is a shape-memory alloy.

10. The method for manufacturing a functional elongate instrument according to claim 9, wherein the shape-memory alloy is a bendable shape-memory alloy.

11. The method for manufacturing a functional elongate instrument according to claim 10, wherein a bending angle of the bendable shape-memory alloy ranges from 0° to 180°.

12. The method for manufacturing a functional elongate instrument according to claim 10, wherein a bending angle of the bendable shape-memory alloy ranges from 0° to 20°.

13. A functional elongate instrument, comprising:
a composite fiber; and
a tip portion at one end portion of the composite fiber, wherein
the composite fiber includes
a first wire as an actuator member,
a second wire opposite to the first wire, and
an insulating portion configured to cover the first wire and the second wire,
the tip portion comprises a connection portion configured to electrically connect the first wire and the second wire, and
a diameter of the composite fiber ranges from 0.1 mm to 1 mm or less.

14. The functional elongate instrument according to claim 13, wherein the actuator member is a shape-memory alloy.

15. The functional elongate instrument according to claim 14, wherein the actuator member is a bendable shape-memory alloy.

16. The functional elongate instrument according to claim 15, wherein a bending angle of the bendable shape-memory alloy ranges from 0° to 180°.

17. The functional elongate instrument according to claim 15, wherein a bending angle of the bendable shape-memory alloy ranges from 0° to 20°.

18. The functional elongate instrument according to claim 15, further comprising:
a plastic conductive wire rod covered with the insulating portion; and
a third wire that is covered with the insulating portion and is opposite to the plastic conductive wire rod, wherein
the third wire is made of Ag, and
the third wire comprises an electrode containing AgCl at a tip thereof.

19. The functional elongate instrument according to claim 15, further comprising:
a third wire covered with the insulating portion; and
a fourth wire that is covered with the insulating portion and is opposite to the third wire.

20. The functional elongate instrument according to claim 13, wherein the insulating portion is made of thermoplastic resin.

21. The functional elongate instrument according to claim 20, wherein the thermoplastic resin is polycarbonate, polystyrene, polyetherimide, or polysulfone.

22. The functional elongate instrument according to claim 13, comprising a hollow portion extending in a longitudinal direction of the composite fiber.
